(19) United States
(12) Patent Application Publication
(10) Pub. No.: US 2003/0175223 A1
(43) Pub. Date: Sep. 18, 2003

(54) Waterborne colored and clear fingernail polish

(76) Inventor: Dominck D. Razzano, Virginia Gardens, FL (US)

Correspondence Address: Robert M. Downey, ROBERT M. DOWNEY, P.A., Suite 350, 150 E. Palmetto Park Road, Boca Raton, FL, 33432, US

(21) Appl. No.: 10/100,257

(22) Filed: Mar. 15, 2002

(51) Int. Cl. A61K 7/04

(52) U.S. Cl. 424/61

(57) ABSTRACT

A waterborne composition for application to fingernails and toenails includes an emulsified resin binder, coalescents, colored pigments, and additives. In a preferred embodiment, the emulsified resin binder is a phenyl ethylene-acrylic copolymer which is available in many different glass transition temperatures (Tg°c.=30° to 100°), each of which have different physical and chemical properties. The formulas for each specific Tg°c. vary in the types of polyol blends of acrylic copolymers. Although other values of Tg°c. may produce fair results, it was found that good results were obtained when a blend of Tg°c.=25 or lower (for flexibility) and Tg°c.=40 or higher (for hardness) were used.

### BACKGROUND OF THE INVENTION

1. Field of the Invention

This invention relates to waterborne clear and pigmented nail polish and, more particularly, to a waterborne organic resin composition for nail polish comprising an emulsified non-toxic binder and a non-toxic pigment.

2. Discussion of the Related Art

The nail polishes used today are based on nitrocellulose as the resin binder and a number of compounds which are considered toxic, including formaldehyde and toluene. Also a high amount (70%) of flammable solvents which have strong odors and various degrees of toxicity are necessary for solubizing the nitrocellulose. The present invention was developed to eliminate all the disadvantages of the organic solvent system. More specifically, the nail polish composition of the present invention uses water as the solvent and phenylethylene-acrylic copolymer (non-toxic) to replace the nitrocellulose, thereby eliminating the need for organic flammable solvents and strong odor organic solvents.

### ADVANTAGES OF THE INVENTION

The waterborne fingernail polish composition of the present invention has the following advantages:

1. Non-flammable

2. Odorless.

3. Non-toxic

4. Minimal effect on the ozone layer.

5. Lower insurance rates for shipping due to its non-flammability.

6. Simpler to manufacture than present nitrocellulose solvent type

7. No allergic reaction (non allergenic)

8. Water based fingernail polish eliminates danger of explosions during manufacturing.

### SUMMARY OF THE INVENTION

The present invention is directed to a waterborne clear or colored pigment liquid applied by brush or spray to fingernails, toenails and artificial nails. The waterborne fingernail polish composition includes an emulsified resin binder, coalescents, and colored pigments. In a preferred embodiment, the emulsified resin binder is a phenyl ethylene-acrylic co-polymer resin.

In use, one or two coats of the colored pigmented composition are applied to the nail surface and allowed to dry for 3-5 minutes. To protect the applied film of colored fingernail polish, one or two coats of clear waterborne composition are applied, allowing 10-20 minutes of drying time between application of the clear coats.

### DETAILED DESCRIPTION OF THE PREFERRED EMBODIMENT

The waterborne organic resin composition of the present invention is intended for application to fingernails, toenails and acrylic artificial nails. The composition comprises an emulsified non-toxic binder in combination with one or more non-toxic pigments which are approved by the U.S. Food and Drug Administration for use in nail polish. The composition further includes coalescents which function to fuse together precipitates (i.e. binder resin particles) into a uniform, smooth and level film when coats of the composition are applied to the surface of fingernails, toenails, and acrylic artificial nails and allowed to air dry.

In a preferred embodiment, the emulsified non-toxic binder comprises a blend of phenyl ethylene acrylic copolymer emulsions consisting of a combination of two different glass transition temperatures. In the preferred embodiment, the glass transition temperatures of the blend are Tg44° c. and Tg25° c. A glass transition temperature of Tg25° c. or lower provides for a softer and more flexible film formation, while a glass transition temperature of Tg44° c. or higher provides for a harder film formation.

The coalescents serve to obtain a continuous film by causing resin gel particles to fuse together. The coalescents further provide good flow-out leveling of film upon drying. In the preferred embodiment, the coalescents include propylene glycol N propyl ether and N methyl-2-pyrroliosine. Other non-toxic coalescents which can be used in the composition include: propylene glycol methyl ether; di-propylene glycol methyl ether; tri-propylene glycol methyl ether; and propylene glycol methyl ether acetate.

The elimination of volatile solvents replaced by water in the composition results in a nonflammable nail polish. Further elimination of volatile solvents provides a more environmentally friendly nail polish product which does not harm the ozone layer.

It should be noted that use of different FDA certified color pigments may require weight adjustments to the components of the composition due to variances in oil absorption and percent concentration values of the pigment portion.

The waterborne nail polish composition consists of two formulas. The first formula is a color-producing resin composition. The second formula is a clear waterborne topcoat resin composition to protect the underlying coats of color resin compositions and to further enhance the gloss of the dried color resin composition.

### EXAMPLE 1

I. Red Color Waterborne Fingernail Polish

| | Ingredients | Lbs | Gals | Supplier |
|---|---|---|---|---|
| 1 | phenyl ethylene acrylic co- | 633.70 | 66.82 | S.C. Johnson |
| | polymer Tg° 44° c | | | |
| 2 | phenyl ethylene acrylic co- | 200.00 | 23.50 | Scott Bader |
| | polymer Tg° 25C | | | |
| 3 | Antifoam Compound | 0.15 | 0.02 | BYK CHEM1 USA |
| | (BYK022) | | | |
| 4 | Anti-settling | 10.50 | 0.78 | RHEOX |
| | SD2 | | | |
| 5 | Propylene glycol-n-propyl | 32.50 | 4.55 | ARCO |
| | ether (coalescent) | | | |
| 6 | N-methyl-2 Pyrrolidone | 32.50 | 4.55 | International |
| | (coalescent) | | | Products |
| 7 | D & C Red 7 calcium | 30.00 | 0.72 | Hilton Davis |
| | LAKE | | | Sun Chemical |
| 8 | Butyl benzyl phthalate | 3.50 | 0.37 | Monsanto Products |
| | (plastizer) | 942.85 | 101.31 | |

Physical constants:

Viscosity 25″-35″#2 Zahn Cup (25° C.)

Wt/Gal 9.30 lbs

% N.V.M. 46.18

### Manufacturing Procedure

| | |
|---|---|
| Lbs | Gals |

A. Preparation of Color Component
Add ingredients according to the following*:

| | | | |
|---|---|---|---|
| 1. | Phenyl ethylene-acrylic copolymer Tg° 44° c | 70.00 | 7.38 |
| 2. | #7 D & C Red #7 Calcium Lake | 30.00 | 0.72 |
| 3. | Anti-foam agent (BYK022( | 0.07 | 0.01 |
| 4. | Anti-settling agent (SD2) | 10.50 | 0.78 |

*Procedure for color dispersion (Cowles) to a number 6-1/2-7 on the
“North Grind Gauge.”

B. Continuing the Process:
In the order below add balance with stirring:

| | | | |
|---|---|---|---|
| 1. | Phenyl ethylene-acrylic copolymer | 563.70 | 59.46 |
| | Tg° 44° c | | |
| 2. | Phenyl ethylene acrylic copolymer Tg° 25C | 200.00 | 23.53 |
| 3. | Antifoam BYK022 | 0.07 | 0.01 |
| 4. | Added Very slowly with high-speed | 32.50 | 4.55 |
| | revolution of Cowles blade: propylene | | |
| | glycol N Butyl ether | | |
| 5. | N-Methyl-2-pyrrolioddone Also added very | 32.50 | 4.55 |
| | slowly with high-speed revolution of | | |
| | Cowles Blade (this prevents seeding) | | |
| 6. | Butul Benzyl Phthalate (Plasticizer) | 3.50 | 0.37 |
| | | 942.84 | 101.36 |

### EXAMPLE 2

II. Preparation of the Waterborne Clear Top Coat

| | Ingredients | Lbs | Gals |
|---|---|---|---|
| 1. | Phenyl ethylene acrylic copolymer Tg44° C. | 833.70 | 90.32 |
| 2. | Antifoam compound BYK022 | .15 | .02 |
| 3. | Propylene glycol N Propylether | 32.50 | 4.55 |
| 4. | N-Methyl-2-Pyrrolidone | 32.50 | 4.55 |
| 5. | Polyethylene #2212 | 15.00 +UZ,28/32 1.83 | |
| | | 913.85 | 101.27 |

### Manufacturing Procedure

Add all ingredients in order listed above, with stirring continuously. In steps 3 and 4, add very slowly with high-speed stirring to avoid “seeding.”

Physical constants:

Viscosity=20″-30″ N02 Zahn

Wt/Gal=9.02 lbs

While the instant invention has been shown and described in accordance with preferred and practical embodiments thereof, it is recognized that departures from the instant disclosure are contemplated within the spirit and scope of the present invention, as defined in the following claims under the doctrine of equivalents.

What is claimed is:

1. A nail polish composition comprising:
an emulsified non-toxic binder;
at least one coalescent; and
a non-toxic pigment portion.

2. The nail polish composition as recited in claim 1 wherein said emulsified non-toxic binder comprises a blend of phenyl ethylene acrylic copolymer emulsions having at least two different glass transition temperatures.

3. The nail polish composition as recited in claim 2 wherein said phenyl ethylene acrylic copolymer emulsions in said blend provide a first glass transition temperature Tg44° c. or higher and a second glass transition temperature of Tg25° c. or lower.

4. The nail polish composition as recited in claim 1 wherein said at least one coalescent is selected from the group consisting of:
propylene glycol N propyl ether;
N methyl-2-pyrroliosine;
propylene glycol methyl ether;
di-propylene glycol methyl ether;
tri-propylene glycol methyl ether; and
propylene glycol methyl ether acetate.

5. The nail polish composition as recited in claim 1 comprising at least two coalescents.

6. The nail polish composition as recited in claim 5 wherein said at least two coalescents include propylene glycol N propyl ether and N methyl-2-pyrroliosine.

7. A nail polish composition comprising:
an emulsified non-toxic binder; and
at least one coalescent.

8. The nail polish composition as recited in claim 7 wherein said emulsified non-toxic binder comprises a blend of phenyl ethylene acrylic copolymer emulsions providing at least two different glass transition temperatures.

9. The nail polish composition as recited in claim 8 wherein said blend of polyethylene acrylic copolymer emulsions provides a first glass transition temperature of Tg25° c. or below and a second glass transition temperature of Tg44° c. or higher.

10. The nail polish composition as recited in claim 8 comprising one or more of said coalescents selected from the group consisting of:
propylene glycol N propyl ether;
N methyl-2-pyrroliosine;
propylene glycol methyl ether;
di-propylene glycol methyl ether;
tri-propylene glycol methyl ether; and
propylene glycol methyl ether acetate.

11. A nail polish composition comprising:
an emulsified non-toxic binder comprising a blend of phenyl ethylene acrylic copolymer emulsions providing at least two different glass transition temperatures;
a blend of coalescents including propylene glycol N propyl ether and N methyl-2-pyrroliosine; and
a non-toxic pigment portion.
